(19) United States
(12) Patent Application Publication
(10) Pub. No.: US 2008/0183020 A1
(43) Pub. Date: Jul. 31, 2008

(54) Catalytic conversion of alcohols to aldehydes and ketones

(75) Inventor: Melvin Keith Carter, Lincoln, CA (US)

Correspondence Address: Carter Technologies, 2300 Sutter View Lane, Lincoln, CA, 95648, US

(73) Assignee: Carter Technologies, Lincoln, CA (US)

(21) Appl. No.: 11/698,089

(22) Filed: Jan. 26, 2007

(51) Int. Cl. C07C 41/01; C07C 47/00; C07C 49/00

(52) U.S. Cl. 568/303; 568/420; 568/579

(57) ABSTRACT

Catalytic reactions are taught for air or oxygen oxidative chemical conversion of primary alcohols to aldehydes, glycol ethers and related products and secondary alcohols to ketones and related products at ambient pressure. The catalytic process converts ethanol to acetaldehyde and 2-ethoxyethanol, n-propanol to propionaldehyde and its glycol ethers, 2-propanol to acetone, and other reactants to similar products. The catalysts are based on molecular strings of di-, tri- and/or poly- groups of transition metal complexes possessing a degree of symmetry. Laboratory results have demonstrated [manganese (II)]₂, [cobalt (II)]₂, [vanadium (II)]₂ and similar families of catalysts to be effective for oxidative catalytic conversion of primary alcohols to products comprising related aldehydes and glycol ethers, and secondary alcohols to products comprising ketones and related products.

### BACKGROUND

1. Field of Invention

Catalytic chemical processes have been reported for converting alcohols to aldehydes by hydration of alkenes, by controlled air oxidation of hydrocarbon gases and as a by-product from the fermentation industries. Controlled air oxidation of certain gaseous hydrocarbons or alcohols may produce small amounts of aldehydes, however such processes have not been identified as economically viable. The invention disclosed in this application teaches oxidative catalytic conversion of alcohols with air or oxygen to aldehydes, glycol ethers, ketones and other products using mono-metal, di-metal, tri-metal and/or poly-metal backbone or molecular string type transition metal catalysts possessing a degree of symmetry without addition of aggressive chemical oxidizing agents and without addition of other strong chemicals.

2. Description of Prior Art

The chemical process industry has grown to maturity based on petroleum feed stocks. Petroleum is a non-renewable resource that may become unavailable in the next 100 to 150 years. This planet Earth fosters continual growth of numerous carbohydrate based plants including fruits, vegetables and grain food sources plus their supporting cellulosic plant stalks and related natural waste materials for recycle. Grains, corn cobs, the support plant stalks and certain grasses are, in part, subject to bio-fermentation processes producing ethanol and related products. A major industry is rapidly developing in ethanol production by fermentation of bio-mass and much of the product is sold as combustion engine fuel or its additive. Ethanol is becoming more available as a renewable resource and this application teaches its catalytic conversion to valued intermediates for use in the hydrocarbon fuels and chemical process industries.

A number of catalytic chemical processes have been reported converting alcohols to aldehydes as presented here. Acetaldehyde has been produced by hydration of acetylene, by controlled air oxidation of propane and butane from natural gas, and as a by-product from the fermentation industries. It can also be produced by reduction of acetic acid and by carbonylation of methanol. Controlled air oxidation of certain gaseous hydrocarbons may produce small amounts of aldehydes, however such processes have not been identified as economically viable. Acetaldehyde formation by direct air oxidation of ethanol has not previously been productive.

There are several hot tube reactions described in the scientific and patent literature for conversion of gaseous alcohols to a wide range of low concentration products from gasoline type hydrocarbons to aldehydes and ethers. Aldehydes and ketones can be formed by passing alcohol vapors over Cu and its alloys or Ag at 300° C. to 600° C. in the presence of controlled amounts of air. U.S. Pat. No. 6,166,265, issued Dec. 26, 2000, introduced a process for preparation of n-butyraldehyde and/or n-butanol by reacting butadiene with an alcohol at super-atmospheric pressure and elevated temperatures using an acid resin or one of several transition metal oxides. U.S. Pat. No. 6,350,918, issued Feb. 26, 2002, teaches a process for the selective oxidation of alcohols to aldehydes in the vapor phase at 150° C. to 600° C. over oxides of V, Cr, Mo, W or Re in their high oxidation states. Less selective chemistry may oxidize an alcohol to aldehydes, ketones and by products. Aldehydes have also be produced by a chemical exchange where one oxidized organic compound may transfer its oxygen atoms to an alcohol converting it to an aldehyde.

Acetaldehyde has also been produced commercially by oxidation of ethanol in air at 480° C. and super-atmospheric pressure in the presence of a silver catalyst. This has been replaced by the Wacker process for oxidation of ethylene that has been more efficient than the ethanol oxidation route. Both processes start with ethylene. Acetaldehyde has also been produced by the expensive hydration of acetylene on a mercury salt catalyst process. Acetaldehyde can be produced from synthesis gas using a rhodium on silica catalyst at elevated temperature and pressure, but the selectivity to acetaldehyde is poor. Acetaldehyde has also been produced by reacting methanol with synthesis gas at elevated temperature and pressure using a cobalt iodide catalyst with a promoter, however neither the rhodium-nor cobalt iodide-catalyzed process has been practiced commercially. U.S. Pat. No. 6,465,694, issued Oct. 15, 2002, reported conversion of polyethylene glycol to aldehyde derivatives in the presence of potassium carbonate over a Cu, Co, Fe, Ni catalyst in air at 40° C. to 90° C. but un-derivatized aldehyde was not reported. U.S. Pat. No. 6,121,498, issued Sep. 19, 2000, disclosed reduction of carboxylic acid compounds to their respective aldehydes in hydrogen gas over palladium on iron oxide catalyst at temperatures of 250° C. to 400° C. U.S. Pat. No. 5,679,870, issued Oct. 21, 1997, published conversion of ketene in hydrogen gas over Co, Rh, Ir, Ni, Pd or Pt catalyst to aldehydes in a temperature range of 50° C. to 200° C. U.S. Pat. No. 4,351,908, issued Sep. 28, 1982, reported carbonylation from synthesis gas on a rhodium catalyst at 75° C. to 125° C. to form alcohols and aldehydes.

Ketones can be prepared by oxidation of secondary alcohols. The process normally requires a strong oxidizing agent such as potassium permanganate, potassium dichromate or other strong oxidizing agents. The alcohol is oxidized by heating under reflux in acidified solution. For example 2-propanol is oxidized to propanone (acetone) where two atoms of hydrogen are removed for each molecule leaving a carbon-oxygen double-bond. U.S. Pat. No. 4,453,015, issued Jun. 5, 1984, disclosed catalytic conversion of secondary butanols to methyl ethyl ketone over a copper, zinc and chromium catalyst on an alpha alumina support at 1 to over 65 atmospheres pressure and 250° C. to 450° C. No reports were identified disclosing catalytic oxidative conversion of alcohols to aldehydes, glycol ethers or ketones at ambient pressure without strong chemical additives.

The invention disclosed in this application teaches oxidative catalytic conversion of alcohols with air or oxygen to aldehydes, glycol ethers, ketones and other products using mono-metal, di-metal, tri-metal and/or poly-metal backbone or molecular string type transition metal catalysts in a low oxidation state possessing a degree of symmetry without addition of aggressive chemical oxidizing agents and without addition of other strong chemicals. This catalytic process results in high yields of the reported products.

It is an object of this invention, therefore, to provide a molecular string type transition metal catalytic process for air or oxygen oxidative conversion of alcohols to aldehydes and glycol ethers without the use of aggressive chemical oxidizing agents or other strong chemicals.

It is another object of this invention to provide molecular string type catalysts for direct air or oxygen oxidation of alcohols to ketones without the use of aggressive chemical oxidizing agents or other strong chemicals. Other objects of this invention will be apparent from the detailed description thereof that follows, and from the claims.

### SUMMARY OF THE INVENTION

This invention describes oxidative catalytic chemical processes for conversion of alcohols in air or oxygen at ambient pressure to aldehydes, glycol ethers and ketones using transition metal catalysts based primarily on di-metal, tri-metal and/or poly-metal backbone or strings possessing a degree of symmetry.

### DETAILED DESCRIPTION OF THE INVENTION

A process is taught for oxidative catalytic chemical conversion of alcohols to aldehydes, glycol ethers, ketones and related products in the presence of air or oxygen employing transition metal compounds, such as [vanadium]₂, [manganese]₂ or [cobalt]₂ type compounds, for which the transition metals and directly attached atoms possess C₄ᵥ, D₄ₕ or D_{2d} point group symmetry. These catalysts have been designed based on a formal theory of catalysis, and the catalysts have been produced, and tested to prove their activity. The theory of catalysis rests upon a requirement that a catalyst possess a single metal atom or a molecular string such that transitions from one molecular electronic configuration to another be barrier free so reactants may proceed freely to products as driven by thermodynamic considerations. Catalysts effective for chemical conversion of alcohols to products can be made from mono-metal, di-metal, tri-metal and/or poly-metal backbone or molecular string type compounds of the transition metals comprising titanium, vanadium, chromium, manganese, iron, cobalt, nickel, copper, zirconium, niobium, molybdenum, ruthenium, rhodium, palladium, silver, hafnium, tantalum, tungsten, rhenium, osmium, iridium, platinum, gold and/or combinations thereof. These catalysts are made in the absence of oxygen so as to produce compounds wherein the oxidation state of the transition metal is low, typically monovalent or divalent although trivalent metal catalysts may also be produced. Anions employed for these catalysts comprise fluoride, chloride, bromide, iodide, cyanide, isocyanate, thiocyanate, sulfate, phosphate, borate, oxide, hydroxide, oxalate, acetate, organic chelating agents and/or other groups. Mixed transition metal compounds have also been found to be effective catalysts for oxidative chemical conversions.

The catalysts act on primary alcohols in the presence of air or oxygen effectively removing terminal hydrogen atoms forming aldehydes under conditions of relatively low temperature and a relatively high air flow rate. Primary alcohols are catalytically oxidized to glycol ethers under conditions of higher temperature and a relatively low air flow rate. Secondary alcohols are catalytically oxidized to ketones under most conditions at somewhat higher temperatures. For example ethanol is catalytically oxidized to acetaldehyde using a cobalt (II) oxalate catalyst on a silica alumina support in a temperature range of 125° C. to 200° C. while n-propanol is catalytically converted to propionaldehyde and its glycol ethers under similar conditions. By contrast 2-propanol is catalytically converted to acetone at temperatures above 150° C.

### Description of Catalyst Preparations And Chemical Conversions

Catalyst preparation was conducted using nitrogen purging and/or nitrogen blanketing to minimize or eliminate air oxidation of the transition metal compounds during preparation. Transition metal catalysts, effective for ambient pressure conversion of alcohols and other hydroxy substituted organic compounds, can be produced by combining transition metal salts in their lowest standard oxidation states with other reactants. Thus, such transition metal catalysts can be made by partially reacting transition metal (I or II) acetates, chlorides, bromides, iodides, sulfates, phosphates, borates, cyanides or similar compounds with other transition metal (I or II) compounds and chelates or by forming transition metal compounds in a reduced state by similar means where mono-, di-, tri- and/or poly-metal compounds result. Some examples follow.

### EXAMPLE 1

The Co₂(C₂H₂O₄)₂ catalyst was prepared in a nitrogen atmosphere by addition of 0.249 gram of cobalt (II) acetate, dissolved in 3 mL of nitrogen purged water, to 15 grams of ⅛ inch diameter alumina silicate cylinders and evaporating to dryness. To this was added 0.433 gram of potassium hydrogen oxalate, dissolved in 15 mL of nitrogen purged water, and the resultant product was heated to approximately 125° C. until dry.

### EXAMPLE 2

The Mn₂(C₂H₂O₄)₂ catalyst was prepared in a nitrogen atmosphere by addition of 0.0989 gram of manganese (II) chloride, dissolved in 3 mL of nitrogen purged water, to 15 grams of ⅛ inch diameter alumina silicate cylinders and evaporating to dryness. To this was added 0.216 gram of potassium hydrogen oxalate, dissolved in 15 mL of nitrogen purged water, and the resultant was heated to approximately 125° C. until dry.

Chemical conversion to aldehydes and ketones was conducted as described. The catalyst was loaded into a stainless steel tube reactor and maintained at its operating temperature. Air was supplied by means of a gas pump, its flow rate was monitored by a gas flow meter, ethanol was delivered by means of a syringe pump and injected onto the catalyst. Resulting products were collected using a cold trap and identified by means of a wet chemical indicator. Ethanol was injected at a rate of 0.20 mL/minute and air was supplied at rates of 0.20 L/minute to 1 L/minute during the reactions at temperatures in the range of 125° C. to 200° C.

### EXAMPLE 3

Air was supplied at a rate of 1 L/minute to a cobalt oxalate catalyst in a reactor controlled at a temperature of 125° C. while ethanol was supplied at a rate of 0.20 mL/minute. A majority of acetaldehyde was produced. Air was also supplied at a rate of 1 L/minute to a cobalt oxalate catalyst controlled at temperatures of 150° C. and 175° C. while ethanol was supplied at a rate of 0.20 mL/minute. Again a majority of acetaldehyde was produced.

### EXAMPLE 4

Air was supplied at a rate of 0.8 L/minute to a cobalt oxalate catalyst in a reactor controlled at a temperature of 130° C. while ethanol was supplied at a rate of 0.20 mL/minute. A majority of acetaldehyde was produced. Air was also supplied at a rate of 0.8 L/minute to a cobalt oxalate catalyst controlled at temperatures of 150° C. and 190° C. to 200° C. with ethanol supplied at a rate of 0.20 mL/minute. Again a majority of acetaldehyde was produced. The same process was conducted at an air flow rate of 0.67 L/minute and catalyst temperatures of 135° C., 175° C. and 202° C. producing a majority of acetaldehyde.

### EXAMPLE 5

Air was supplied at rates of 0.20 L/minute, 0.35 L/minute and 0.50 L/minute to a cobalt oxalate catalyst in a reactor controlled at a temperature of 125° C. while ethanol was supplied at a rate of 0.20 mL/minute. A majority of acetaldehyde was produced. Again air was supplied at rates of 0.20, 0.35 and 0.50 L/minute to a cobalt oxalate catalyst controlled at temperatures of 162° C. and 200° C. while ethanol was supplied at a rate of 0.20 mL/minute. A mixture of acetaldehyde and ethoxyethanol was produced.

### EXAMPLE 6

Isopropyl alcohol was injected on to a cobalt oxalate on silica-alumina catalyst packed tube reactor in the temperature range of 175° C. to 200° C. at air flow rates of 0.20, 0.35 and 0.50 L/minute where acetone was produced.

### REFERENCES CITED

### U.S. Patent Documents

Patent No. Issue Date Author Comments
6,465,694 10/15/2002 M Baudys, F Liu, S W Kim PEG+K₂CO₃ on Cu, Co, Fe, Ni catalyst in air to form aldehyde
6,121,498 9/19/2000 G C Tustin, L S Depew, N A RCO₂H+H₂ on Pd/Fe₂O₃ catalyst to form aldehyde Collins
5,723,679 3/3/1998 A Keshavarmja, J V Samuel, A V t-BuOH to MEK on CuO/SiO₂ at 210° C.-375° C., Ramaswamy 1 atm-5 atm
5,679,870 10/21/1997 G C Tustin, L S Depew Ketene+H₂ on Co, Rh, Ir, Ni, Pd or Pt catalyst to form aldehyde
4,380,673 4/19/1983 J-P Bournonville, R Snappe, J s-ROH on Cu, Ag, Au, Sn, Pb, Zn, Cd, In, Ge/Raney Ni to Miquel, G Martino form ketone (185° C.-210° C.)

What is claimed:

1. Catalytic oxidative chemical conversion of alcohols with air or oxygen to aldehydes, glycol ethers, ketones and related products on transition metal compounds comprising low valent titanium, vanadium, chromium, manganese, iron, cobalt, nickel, copper, zirconium, niobium, molybdenum, ruthenium, rhodium, palladium, silver, hafnium, tantalum, tungsten, rhenium, osmium, iridium, platinum, gold and/or combinations thereof that are not oxides.

2. Catalytic oxidative chemical conversion of alcohols with air or oxygen to aldehydes, glycol ethers and ketones for reactants comprising ethanol to acetaldehyde and 2-ethyoxy ethanol, n-propanol to propionaldehyde and 2-propanoxy propanol, 2-propanol to acetone and related products on transition metal compounds comprising low valent titanium, vanadium, chromium, manganese, iron, cobalt, nickel, copper, zirconium, niobium, molybdenum, ruthenium, rhodium, palladium, silver, hafnium, tantalum, tungsten, rhenium, osmium, iridium, platinum, gold and/or combinations thereof that are not oxides.

3. Catalytic oxidative chemical conversion of alcohols with air or oxygen to aldehydes and related products on transition metal compounds comprising low valent titanium, vanadium, chromium, manganese, iron, cobalt, nickel, copper, zirconium, niobium, molybdenum, ruthenium, rhodium, palladium, silver, hafnium, tantalum, tungsten, rhenium, osmium, iridium, platinum, gold and/or combinations thereof that are not oxides at temperatures in the range of 125° C. to 200° C. for aldehydes and glycol ethers, and 125° C. to 180° C. for ketones.

4. Catalytic oxidative chemical conversion of alcohols with air or oxygen to aldehydes, glycol ethers and ketones for reactants comprising ethanol to acetaldehyde and 2-ethyoxy ethanol, n-propanol to propionaldehyde and 2-propanoxy propanol, 2-propanol to acetone and related products on transition metal compounds comprising low valent titanium, vanadium, chromium, manganese, iron, cobalt, nickel, copper, zirconium, niobium, molybdenum, ruthenium, rhodium, palladium, silver, hafnium, tantalum, tungsten, rhenium, osmium, iridium, platinum, gold and/or combinations thereof that are not oxides at temperatures in the range of 125° C. to 200° C. for aldehydes and glycol ethers, and 125° C. to 180° C. for ketones.
